(19) United States
(12) Patent Application Publication
(10) Pub. No.: US 2003/0170191 A1
(43) Pub. Date: Sep. 11, 2003

(54) Hair growth stimulant compositions with sustained action

(76) Inventors: Koji Imamura, Tokyo (JP); Rumi Ochiai, Tokyo (JP); Takako Okajima, Tokyo (JP); Susumu Morioka, Tokyo (JP); Taro Horie, Tokyo (JP)

Correspondence Address: SUGHRUE MION, PLLC, 2100 PENNSYLVANIA AVENUE, N.W., WASHINGTON, DC, 20037, US

(21) Appl. No.: 10/240,599

(22) PCT Filed: Apr. 5, 2001
(86) PCT No.: PCT/JP01/02941

(30) Foreign Application Priority Data
Apr. 7, 2000 (JP) 2000-106498

(51) Int. Cl. A61K 7/06; A61K 31/513

(52) U.S. Cl. 424/70.1; 514/269

(57) ABSTRACT

Minoxidil preparations having a sustained action by which a fully satisfactory effect of stimulating hair growth can be obtained when applied once a day.

Namely, hair growth stimulant compositions with a sustained action, which comprise (A) 2 to 6% by mass of minoxidil and (B) 5 to 30% by mass of a mixture of 1,3-butylene glycol or dipropylene glycol with glycerol blended at a weight ratio of 10:1 to 1:4, exert an excellent effect of stimulating hair growth.

### TECHNICAL FIELD

The present invention relates to a hair growth stimulant composition with sustained action (sustained release hair growth composition) comprising minoxidil as an active component, and more particularly to a sustained release hair growth composition possessing a prolonged effect achieved by retaining minoxidil in the dermis of the scalp for a long period of time.

### BACKGROUND ART

The chemical name for minoxidil is 6-(1-piperidinyl)-2,4-pyrimidinediamine-3-oxide. The adaptation of minoxidil as a hair growth agent is disclosed in U.S. Pat. No. 4,139,619. Due to the excellent hair growth obtained by the topical application of minoxidil, hair growth agents containing minoxidil (hereinafter referred to as “minoxidil preparations”) have been widely accepted and their sales volume is record breaking.

Because minoxidil preparations have to be applied several times a day, application tends to be forgotten during busy times. As a result, there are instances where a sufficient hair growth effect is not obtained. Research has been conducted to obtain a sustained release minoxidil preparation having a sufficient hair growth effect through a once-per-day application to coincide with the rhythm of human life, however there are currently very few sustained release minoxidil preparations that can satisfactorily accomplish this.

The present invention was accomplished in light of this situation to obtain a sustained release minoxidil preparation having an excellent hair growth effect through a once-per-day application.

### DISCLOSURE OF THE INVENTION

To investigate the conditions under which the percutaneously absorbed minoxidil was stably retained in the dermis of the scalp, the present inventors examined the composition of the minoxidil preparation. As a result, the inventors have discovered that the above objective can be accomplished using a solution of a specific dihydric alcohol and a specific trihydric alcohol, thereby completing the present invention.

Accordingly, an object of the present invention is to provide a sustained release hair growth composition comprising (A) 2 to 6% by mass of minoxidil and (B) 5 to 30% by mass of a mixture of 1,3-butylene glycol or dipropylene glycol with glycerol blended at a weight ratio of 10:1 to 1:4.

### BRIEF DESCRIPTION OF THE DRAWING

FIG. 1 is a graph showing the measurement results of the concentration of minoxidil in the blood for the preparations of the present invention and a comparative preparation.

### DETAILED DESCRIPTION OF THE PREFERRED EMBODIMENT

The sustained release hair growth composition of the present invention is produced by mixing 5 to 30% by mass (hereinafter simply referred to as “%”) of a mixture of 1,3-butylene glycol or dipropylene glycol with glycerol blended at a weight ratio of 10:1 to 1:4 (component (B)) and 2 to 6% of the active component minoxidil (component (A)), respectively for the total amount of the composition.

The sustained release hair growth composition even more preferably contains 3 to 6% of the component (A) (minoxidil) In view of sustained release action, the amount of the component (B) is preferably 10% or more and even more preferably 14% or more. In view of feeling during use, the amount is preferably 20% or less. Also, in view of the solubility of minoxidil and feeling during use of the hair growth composition, the incorporated amount of the component (B) (weight ratio; hereinafter the same) in regard to the component (A) is even more preferably in a range of about 1:1 to 1:5.

In the present invention, a mixture of 1,3-butylene glycol or dipropylene glycol as the dihydric alcohol and glycerol as the trihydric alcohol with a weight ratio of 10:1 to 1:4 must be used as the component (B). 1,3-butylene glycol or dipropylene glycol and glycerol at a weight ratio of 6:1 to 1:1 are even more preferable. The dihydric alcohol of the component (B) may be a mixture of 1,3-butylene glycol and dipropylene glycol.

The sustained release hair growth composition of the present invention is composed of the above composition, however, when necessary ethanol and water may be combined with the composition. In this instance, a preferable range for the incorporated amount of ethanol is about 40 to 82% and a preferable range for the incorporated amount of water is 5 to 30%.

The sustained release-hair growth composition of the present invention is preferably adjusted to a pH of 5.5 to 9.5, and even more preferably to a pH of 5.5 to 6.5. An acid and/or base may be used as the pH adjustor for adjusting the pH. As preferable examples of the pH adjustor, citric acid, hydrochloric acid, lactic acid, phosphoric acid, and the like can be given.

In addition to the above components, other necessary active components and supplementary components may be added to the sustained release hair growth composition of the present invention. As medicinal components that are preferably added to and combined with the sustained release hair growth composition of the present invention, components selected from the group consisting of menthol, vitamin E acetate, pantothenylethylether, hinokitiol, glycyrrhetic acid, and diphenhydramine hydrochloride can be given (hereinafter referred to as “optional components”). Of these optional components, menthol and pantothenylethylether are preferable in view of sustained release action.

There are no particular limitations to the amount of these optional components to be added. This can be decided experimentally while taking the feeling during use and stability of minoxidil or solvent-type components into consideration. For example, the final composition preferably contains about 0.2 to 0.4% of menthol, a particularly preferable optional component.

In addition to the above components, the sustained release hair growth composition of the present invention may contain various active components and supplementary components normally used in topical preparations in an amount that does not harm the effect of the present invention. Examples of these components include fillers, vasodilators (carpronium chloride, benzyl nicotinate, swertia herb extract, panax ginseng extract, capsicum tincture, and the like), antihistamines (isothipendyl hydrochloride and the like), anti-inflammatory agents (guaiazulene and the like), keratolytics (urea, salicylic acid, and the like), antimicrobial agents (chlorhexidine gluconate, isopropyl methylphenol, quaternary ammonium salts, piroctone olamine, and the like), humectants (sodium hyaluronate, chondroitin sulfuric acid, and the like), extracts of animals and plants (Taxus cuspidata, Paeonia suffruticosa, Glycyrrhisa uralensis, Hypericum erectum, aconite root, Eriobotrya japonica, Artemisia capillaris, Symphytum officinale, Angelica keiskei, Crocus sativus, Gardeniae fructus, Rosmarinus officinalis, Salvia officinalis, Saussurea lappa, Aristolochia debilis, Lupuli strobilus, placenta, and the like), vitamins (retinol acetate, pyridoxine hydrochloride, ascorbic acid, thiamin nitrate, cyanocobalamin, biotin, and the like), anti-oxidants (dibutylhydroxytoluene, sodium pyrosulfite, tocopherol, sodium edetate, ascorbic acid, isopropyl gallate, and the like), solubilizers (diisopropyl adipate, isopropyl myristate, vegetable oils, animal oils, alkyl glyceryl ethers, hydrocarbons, and the like), metabolic activators, gelling agents (water-soluble high molecular compounds and the like), adhesives, perfumes, refrigerants (mentha oil, camphor, and the like), dyes, and the like.

However, since the addition of surfactants affects the cutaneous absorption of minoxidil and decreases the feeling during use, it is preferable that the sustained release hair growth composition of the present invention does not substantially contain surfactants.

The sustained release hair growth composition of the present invention thus obtained can be used as a suitable topical preparation such as a lotion, aerosol, tonic, cream, ointment, gel, and the like.

### EXAMPLES

The present invention will be described in more detail by examples, which should not be construed as limiting the present invention.

### Example 1

Sustained Release Hair Growth Lotion:

2 g of minoxidil, 7 g of 1,3-butylene glycol, 3 g of glycerol, and 60.05 g of ethanol were mixed and stirred until dissolved. Next, purified water was added to the mixture to obtain 100 ml of a lotion (preparation 1).

### Example 2

Using the components shown in Tables 1-9, lotion preparations 2-38 (products of the present invention) were prepared in the same manner as in Example 1. Likewise, the lotion preparations 39-41 (comparative products) were prepared using the components shown in Table 10. Using the following test method, the concentration of minoxidil in the blood for the lotion products of the present invention and the comparative lotion products was measured. These results showed that minoxidil in the products of the present invention was released more slowly and retained for a longer period of time.

(Composition)

TABLE 1

| | Preparation 1 | Preparation 2 | Preparation 3 |
|---|---|---|---|
| minoxidil | 2 g | 2 g | 2 g |
| 1,3-butylene glycol | 7 g | 6 g | 5 g |
| glycerol | 3 g | 4 g | 5 g |
| ethanol | 60.05 g | 60.05 g | 60.05 g |
| purified water | balance* | balance* | balance* |
| pH | 8.6 | 8.7 | 8.7 |

*The total amount of the preparation is 100 ml

TABLE 2

| | Preparation 4 | Preparation 5 | Preparation 6 | Preparation 7 |
|---|---|---|---|---|
| minoxidil | 3 g | 3 g | 5 g | 5 g |
| 1,3-butylene | 6 g | 5 g | 10 g | 15 g |
| glycol | | | | |
| glycerol | 4 g | 5 g | 10 g | 15 g |
| ethanol | 60.05 g | 60.05 g | 51.47 g | 42.89 g |
| purified water | balance* | balance* | balance* | balance* |
| pH | 8.8 | 8.8 | 9.0 | 8.9 |

*The total amount of the preparation is 100 ml

TABLE 3

| | Prepara- | Prepara- | Prepara- | Prepara- | Prepara- |
|---|---|---|---|---|---|
| | tion 8 | tion 9 | tion 10 | tion 11 | tion 12 |
| minoxidil | 3 g | 3 g | 3 g | 5 g | 5 g |
| 1,3-butylene | 7 g | 6 g | 5 g | 17 g | 15 g |
| glycol | | | | | |
| glycerol | 3 g | 4 g | 5 g | 3 g | 5 g |
| ethanol | 60.05 g | 60.05 g | 60.05 g | 51.47 g | 51.47 g |
| citric acid | proper | — | — | — | 1.0 g |
| | amount | | | | |
| phosphoric | — | proper | — | — | — |
| acid | | amount | | | |
| lactic acid | — | — | proper | — | — |
| | | | amount | | |
| hydrochloric | — | — | — | proper | — |
| acid | | | | amount | |
| sodium | — | — | — | — | proper |
| hydroxide | | | | | amount |
| purified | balance* | balance* | balance* | balance* | balance* |
| water | | | | | |
| pH | 6.0 | 6.0 | 5.5 | 5.6 | 6.5 |

*The total amount of the preparation is 100 ml

TABLE 4

| | Prepara- | Prepara- | Prepara- | Prepara- | Prepara- |
|---|---|---|---|---|---|
| | tion 13 | tion 14 | tion 15 | tion 16 | tion 17 |
| minoxidil | 5 g | 5 g | 5 g | 5 g | 5 g |
| 1,3-butylene | 18 g | 15 g | 13 g | 12 g | 10 g |
| glycol | | | | | |
| glycerol | 3 g | 5 g | 7 g | 8 g | 10 g |
| ethanol | 60.05 g | 60.05 g | 60.05 g | 60.05 g | 60.05 g |
| citric acid | proper | — | — | — | — |
| | amount | | | | |
| phosphoric | — | proper | — | — | — |
| acid | | amount | | | |
| lactic acid | — | — | proper | — | 1.0 g |
| | | | amount | | |
| hydrochloric | — | — | — | proper | — |
| acid | | | | amount | |
| sodium | — | — | — | — | proper |
| hydroxide | | | | | amount |
| purified | balance* | balance* | balance* | balance* | balance* |
| water | | | | | |
| pH | 6.0 | 6.0 | 5.5 | 5.6 | 6.5 |

*The total amount of the preparation is 100 ml

TABLE 5

| | Preparation 18 | Preparation 19 | Preparation 20 |
|---|---|---|---|
| minoxidil | 2 g | 2 g | 2 g |
| dipropylene glycol | 7 g | 6 g | 5 g |
| glycerol | 3 g | 4 g | 5 g |
| ethanol | 60.05 g | 60.05 g | 60.05 g |
| purified water | balance* | balance* | balance* |
| pH | 8.6 | 8.7 | 8.6 |

*The total amount of the preparation is 100 ml

TABLE 6

| | Preparation | Preparation | Preparation | Preparation |
|---|---|---|---|---|
| | 21 | 22 | 23 | 24 |
| minoxidil | 3 g | 3 g | 5 g | 5 g |
| dipropylene | 6 g | 5 g | 10 g | 15 g |
| glycol | | | | |
| glycerol | 4 g | 5 g | 10 g | 15 g |
| ethanol | 60.05 g | 60.05 g | 51.47 g | 42.89 g |
| purified water | balance* | balance* | balance* | balance* |
| pH | 8.8 | 8.8 | 8.9 | 9.0 |

*The total amount of the preparation is 100 ml

TABLE 7

| | Prepara- | Prepara- | Prepara- | Prepara- | Prepara- |
|---|---|---|---|---|---|
| | tion 25 | tion 26 | tion 27 | tion 28 | tion 29 |
| minoxidil | 3 g | 3 g | 3 g | 5 g | 5 g |
| dipropylene | 7 g | 6 g | 5 g | 17 g | 15 g |
| glycol | | | | | |
| glycerol | 3 g | 4 g | 5 g | 3 g | 5 g |
| ethanol | 60.05 g | 60.05 g | 60.05 g | 51.47 g | 51.47 g |
| citric acid | proper | — | — | — | 1.0 g |
| | amount | | | | |
| phosphoric | — | proper | — | — | — |
| acid | | amount | | | |
| lactic acid | — | — | proper | — | — |
| | | | amount | | |
| hydrochloric | — | — | — | proper | — |
| acid | | | | amount | |
| sodium | — | — | — | — | proper |
| hydroxide | | | | | amount |
| purified | balance* | balance* | balance* | balance* | balance* |
| water | | | | | |
| pH | 6.0 | 6.0 | 5.5 | 5.6 | 6.5 |

*The total amount of the preparation is 100 ml

TABLE 8

| | Prepara- | Prepara- | Prepara- | Prepara- | Prepara- |
|---|---|---|---|---|---|
| | tion 30 | tion 31 | tion 32 | tion 33 | tion 34 |
| minoxidil | 5 g | 5 g | 5 g | 5 g | 5 g |
| dipropylene | 18 g | 15 g | 13 g | 12 g | 10 g |
| glycol | | | | | |
| glycerol | 3 g | 5 g | 7 g | 8 g | 10 g |
| ethanol | 60.05 g | 60.05 g | 60.05 g | 60.05 g | 60.05 g |
| citric acid | proper | — | — | — | — |
| | amount | | | | |
| phosphoric | — | proper | — | — | — |
| acid | | amount | | | |
| lactic acid | — | — | proper | — | 1.0 g |
| | | | amount | | |
| hydrochloric | — | — | — | proper | — |
| acid | | | | amount | |
| sodium | — | — | — | — | proper |
| hydroxide | | | | | amount |
| purified | balance* | balance* | balance* | balance* | balance* |
| water | | | | | |
| pH | 6.0 | 6.0 | 5.5 | 5.6 | 6.5 |

*The total amount of the preparation is 100 ml

TABLE 9

| | Preparation | Preparation | Preparation | Preparation |
|---|---|---|---|---|
| | 35 | 36 | 37 | 38 |
| minoxidil | 5 g | 5 g | 5 g | 5 g |
| dipropylene | 18 g | 15 g | 10 g | 12 g |
| glycol | | | | |
| glycerol | 3 g | 5 g | 5 g | 6 g |
| ethanol | 60.05 g | 60.05 g | 60.05 g | 60.05 g |
| citric acid | proper | — | — | — |
| | amount | | | |
| phosphoric acid | — | — | proper | — |
| | | | amount | |
| lactic acid | — | — | — | amount |
| | | | | amount |
| hydrochloric | — | 0.2 g | — | — |
| acid | | | | |
| sodium | — | proper | — | — |
| hydroxide | | amount | | |
| L-menthol | 0.3 g | 0.3 g | 0.3 g | 0.3 g |
| vitamin E | 0.08 g | 0.08 g | — | — |
| acetate | | | | |
| pantothenyl- | 1 g | 1 g | 1 g | 1 g |
| ethylether | | | | |
| hinokitiol | 0.05 g | 0.05 g | — | — |
| glycyrrhetic | 0.1 g | 0.1 g | — | — |
| acid | | | | |
| di- | 0.1 g | 0.1 g | — | — |
| phenhydramine | | | | |
| hydrochloride | | | | |
| purified water | balance* | balance* | balance* | balance* |
| pH | 6.0 | 6.1 | 6.0 | 6.0 |

*The total amount of the preparation is 100 ml

TABLE 10

| | Preparation 39 | Preparation 40 | Preparation 41 |
|---|---|---|---|
| minoxidil | 2 g | 3 g | 5 g |
| 1,3-butylene glycol | 10 g | 12 10 g | 10 g |
| glycerol | — | — | — |
| ethanol | 60.05 g | 60.05 g | 60.05 g |
| citric acid | — | — | proper amount |
| purified water | balance* | balance* | balance* |
| pH | 8.7 | 8.8 | 6.0 |

*The total amount of the preparation is 100 ml

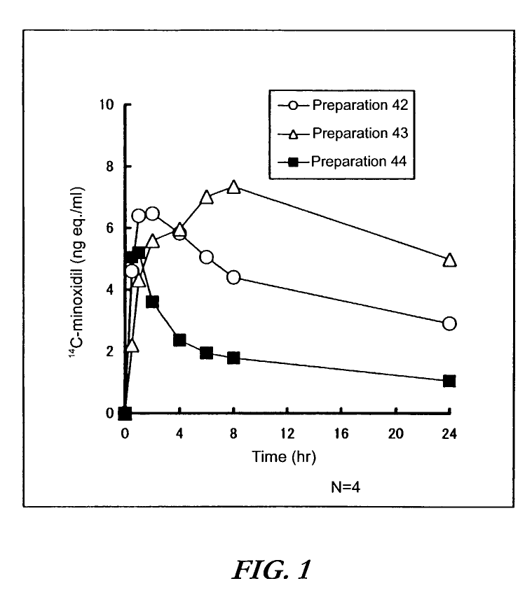

Using the components shown in Table 11, lotion preparations 42 and 43 (products of the present invention) were prepared in the same manner as in Example 1. Likewise, lotion preparation 44 (comparative product) was prepared using the components shown in Table 11. The results of measurement of the concentration of minoxidil in the blood for the lotion products of the present invention and the comparative lotion products using the following test method are shown in FIG. 1. These results confirm that the products of the present invention release minoxidil much slower.

TABLE 11

| | Preparation 42 | Preparation 43 | Preparation 44 |
|---|---|---|---|
| minoxidil | 2 g | 2 g | 2 g |
| 1,3-butylene glycol | 8 g | — | 2 g |
| dipropylene glycol | — | 8 g | — |
| glycerol | 8 g | 8 g | 2 g |
| ethanol | 50 g | 50 g | 50 g |
| phosphoric acid | 0.1 ml | 0.1 ml | — |
| purified water | balance* | balance* | balance* |
| pH | 6.0 | 6.0 | 8.7 |

*The total amount of the preparation is 100 ml

(Test Method)

Male Wistar rats (8 weeks old), whose abdominal hairs were shaved, were positioned supine under ether anesthesia, and 30 μl of each lotion prepared with ¹⁴C-minoxidil was applied on the defined area (2×3 cm) of the abdomen. At each defined time period (0.5, 1, 2, 4, 6, 8, and 24 hours), blood was collected from the jugular vein, and the concentration of minoxidil in the blood was measured using a liquid scintillation counter.

### Example 4

An aerosol was prepared by filling an aerosol can with 30 ml of the lotion prepared in Example 1 and 70 ml of dimethyl ether. In the same manner, an aerosol was prepared using the lotion of Example 2.

1. A sustained release hair growth composition comprising (A) 2 to 6% by mass of minoxidil and (B) 5 to 30% by mass of a mixture of 1,3-butylene glycol or dipropylene glycol with glycerol blended at a weight ratio of 10:1 to 1:4.

2. The sustained release hair growth composition according to claim 1, wherein the weight ratio of 1,3-butylene glycol or dipropylene glycol and glycerol of the component (B) is 6:1 to 1:1.

3. The sustained release hair growth composition according to either claim 1 or 2, wherein the weight ratio of the minoxidil of the component (A) and the mixed solution of the component (B) is 1:1 to 1:5.

4. The sustained release hair growth composition according to any one of claims 1-3, wherein the content of the minoxidil of the component (A) is 3 to 6% by mass.

5. The sustained release hair growth composition according to any one of claims 1-4, wherein the composition has a pH of 5.5 to 9.5.

6. The sustained release hair growth composition according to any one of claims 1-4, wherein the composition has a pH of 5.5 to 6.5.

7. The sustained release hair growth composition according to any one of claims 1-6 further comprising ethanol and water.

8. The sustained release hair growth composition according to claim 7, wherein the content of ethanol is 40 to 82% by mass.

9. The sustained release hair growth composition according to either claim 7 or 8, wherein the content of water is 5 to 30% by mass.

10. The sustained release hair growth composition according to any one of claims 1-9 substantially not containing surfactants.

11. The sustained release hair growth composition according to any one of claims 1-10 containing an acid and/or base as a pH adjustor.

12. The sustained release hair growth composition according to claim 11, wherein the pH adjustor is citric acid, hydrochloric acid, lactic acid, or phosphoric acid.

13. The sustained release hair growth composition according to any one of claims 1-12 further comprising one or more components selected from the group consisting of menthol and pantothenylethylether.

14. The sustained release hair growth composition according to any one of claims 1-13 in the form of a topical lotion.

15. The sustained release hair growth composition according to any one of claims 1-13 in the form of a topical aerosol.

16. The sustained release hair growth composition according to any one of claims 1-13 in the form of a gel.

17. A method of sustaining the effect of a hair growth composition comprising adding 1,3-butylene glycol or dipropylene glycol and glycerol to a hair growth composition containing 2-6% by mass of minoxidil.

18. The method according to claim 17, wherein the content of 1,3-butylene glycol or dipropylene glycol and glycerol is 5 to 30% by mass based on the hair growth composition.

19. The method according to either claim 17 or 18, wherein the ratio of 1,3-butylene glycol or dipropylene glycol and glycerol is 10:1 to 1:4.
